(19) United States
(12) Patent Application Publication
(10) Pub. No.: US 2024/0310359 A1
(43) Pub. Date: Sep. 19, 2024

(54) COMPOSITION FOR PREVENTING OR TREATING SKELETAL MUSCLE DISEASE

(71) Applicant: THE CATHOLIC UNIVERSITY OF KOREA INDUSTRY ACADEMIC COOPERATION FOUNDATION, Seoul (KR)

(72) Inventors: Eun Hui LEE, Seoul (KR); Jun Hee CHOI, Seoul (KR); Seung Yeon JEONG, Seoul (KR); Jooho KIM, Seoul (KR)

(21) Appl. No.: 18/595,964

(22) Filed: Mar. 5, 2024

(30) Foreign Application Priority Data
Mar. 15, 2023 (KR) 10-2023-0033884

(51) Int. Cl. G01N 33/50; A61K 38/17; A61P 21/00

(52) U.S. Cl. CPC G01N 33/502; A61K 38/1709; A61P 21/00; G01N 33/5061; G01N 2333/4703; G01N 2500/10; G01N 2800/10

(57) ABSTRACT

The present invention relates to a pharmaceutical composition for preventing or treating a skeletal muscle disease, a method of screening a drug for the treatment of a skeletal muscle disease, a composition for diagnosing a skeletal muscle disease, and a method of providing information for the diagnosis of a skeletal muscle disease.

### CROSS-REFERENCE TO RELATED APPLICATION

This application claims priority to and the benefit of Korean Patent Application No. 10-2023-0033884, filed on Mar. 15, 2023, the disclosure of which is incorporated herein by reference in its entirety.

### SEQUENCE LISTING

The application contains a Sequence Listing that has been filed electronically, created Mar. 4, 2024, and named “DANA-42899-201_SequenceListing.xml” (10,595 bytes), the contents of which are incorporated by reference herein in their entirety.

### BACKGROUND

### 1. Field of the Invention

The present invention relates to a composition for preventing or treating a skeletal muscle disease or a method of screening the same.

### 2. Discussion of Related Art

Store-operated Ca²⁺ entry (SOCE) is a process of regulating a calcium ion level in cells by detecting the change in calcium level stored in calcium storage in cells. Calcium ions play an important role in the contraction of skeletal muscles, nerve signaling, and many cellular processes, including gene expression. To maintain appropriate levels of calcium ions in cells, SOCE, one of mechanisms to import extracellular calcium ions into the cytosol, is used by cells.

SOCE is caused when the level of calcium stored in the endoplasmic reticulum (the sarcoplasmic reticulum in skeletal muscle cells) decreases. The decrease in the stored calcium activates a protein known as a STIM1 (a calcium sensor), making a calcium channel on the cell membrane open. This allows extracellular calcium ions to enter the cytosol from the extracellular space to increase the cytosolic calcium concentration, allowing various functions in the cell that require calcium to occur smoothly.

SOCE is a process that plays an important role in many cell functions, including cell proliferation, differentiation, and activation. SOCE dysregulation is associated with various diseases including various skeletal muscle diseases, cancer, cardiovascular diseases, and neurological disorders.

Such a change in SOCE may affect the normal function of skeletal muscle cells, causing defects with the contraction and relaxation of skeletal muscles. The decrease in SOCE may lead to a decrease in calcium levels in cells that can damage the contraction of skeletal muscles. Calcium ions bind to troponin C protein, which acts as a signal causing bridge-bonding between actin filaments and a myosin filaments for the contraction of skeletal muscles, thereby playing an important role in the contraction of skeletal muscles. When a cytosolic calcium level is lower than the normal level, the contraction of skeletal muscles may weaken or even fail, leading to skeletal muscle fatigue and weakness.

On the other hand, an excessive increase in SOCE may lead to an increase in a cytosolic calcium level, which negatively affects skeletal muscle cells. An excessively high calcium level in the cytosol, caused by excessive SOCE, may activate a cellular process such as oxidative stress and cell damage which causes the dysfunctions of skeletal muscles and skeletal muscle diseases. In addition, an excessively high calcium level in the cytosol may lead to the inappropriate protease activation, contributing to the degradation of a skeletal muscle proteins resulting the loss of skeletal muscles and abnormal contraction and relaxation of skeletal muscle.

Accordingly, the appropriate regulation of SOCE is important in maintaining the normal functions of skeletal muscles. An excessive increase or decrease in a SOCE level is known to cause the dysfunction of skeletal muscles and skeletal muscle diseases.

Therefore, the inventors identified the mechanism for diagnosing and treating a skeletal muscle-related disease accompanied by the excessive increase or decrease in a SOCE level, and thus completed the present invention.

### SUMMARY OF THE INVENTION

The present invention is directed to providing a method of screening a drug for treating a skeletal muscle disease.

The present invention is also directed to providing a composition for diagnosing a skeletal muscle disease.

The present invention is also directed to providing a method of providing information for the diagnosis of a skeletal muscle disease.

The present invention is also directed to providing a pharmaceutical composition for preventing or treating a skeletal muscle disease.

The inventors found that TRIM32 in differentiated skeletal muscle cells can bind to SERCA1a via its NHL repeats. In addition, the inventors confirmed that, when wild-type TRIM32 is expressed in skeletal muscle cells, it increases a cytosolic calcium level for skeletal muscle contraction and increases the migration of internal calcium for skeletal muscle contraction through binding of TRIM32 to SERCA1a without affecting the degree of differentiation into skeletal muscle cells or the degree of expression of key proteins involved in skeletal muscle contraction and relaxation.

Therefore, according to an aspect of the present invention, there is provided a composition for diagnosing a skeletal muscle disease, which includes an agent for measuring the interaction(s) of TRIM32 and/or NHL repeats with SERCA1a.

According to another aspect of the present invention, there is provided a method of providing information for diagnosing a skeletal muscle disease, which includes measuring the interaction(s) of TRIM32 and/or NHL repeats with SERCA1a from a biological sample isolated from a subject.

According to still another aspect of the present invention, there is provided a method of screening a drug for treating a skeletal muscle disease, which includes treating a biological sample isolated from a subject suspected of having a skeletal muscle disease with a candidate material, and confirming whether the candidate material increases or decreases the interaction(s) of TRIM32 and/or NHL repeats with SERCA1a in skeletal muscle cells.

According to yet another aspect of the present invention, there is provided a pharmaceutical composition for treating a skeletal muscle disease, which includes NHL-Del or a polynucleotide encoding the NHL-Del.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1A illustrates wild-type TRIM32 or NHL repeats, wherein the numbers indicate the amino acid sequence. FIG. 1B shows the result of Coomassie blue staining that confirms the expression of GST-NHL protein (left), the degree of GST-NHL purification (middle), and the binding reaction between a triad sample and GST-NHL (right). FIG. 1C to FIG. 1G show the MALDI-TOF/TOF spectra for bands 1 to 5. FIG. 1H is the results obtained through co-immunoprecipitation using triad samples obtained from rabbit skeletal muscles and SERCA1a antibodies, and immunoblot analysis performed on the obtained samples using SERCA1a and TRIM32 antibodies (top). This drawing also shows the results obtained through co-immunoprecipitation using mouse differentiated skeletal muscle cell lysate solution samples and SERCA1a antibodies, and immunoblot analysis performed on the obtained samples using SERCA1a and TRIM32 antibodies (bottom);

FIG. 2A illustrates wild-type TRIM32 or NHL-Del, wherein the numbers indicate the amino acid sequence. FIG. 2B shows the expression of a vector (vector control), wild-type TRIM32 or NHL-Del in differentiated skeletal muscle cells, confirmed by immunochemistry (white bar: 100 um). FIG. 2C shows the measured width of the thickest part of the differentiated skeletal muscle cell. FIG. 2D shows the degrees of expression of the differentiation marker proteins (MyoD and myogenin proteins) and the housekeeping protein in all cells, a-actin, confirmed by immunoblot analysis;

FIG. 3 is the result of immunoblot analysis performed to compare expression levels of key proteins that mediate skeletal muscle contraction and relaxation, such as RyR1, DHPR, SERCA1a, TRIM32 (endogenous), MG53, and CASQ1;

FIG. 4A is a bar graph showing a calcium amount stored in the sarcoplasmic reticulum in mouse differentiated skeletal muscle cells expressing wild-type TRIM32 or NHL-Del. FIG. 4B shows the relative degree of calcium release from the sarcoplasmic reticulum to the cytosol in mouse differentiated skeletal muscle cells by treatment with caffeine. FIG. 4C shows the relative amount of calcium released from the sarcoplasmic reticulum to the cytosol in mouse differentiated skeletal muscle cells by treatment with KCl; and

FIG. 5 shows the external calcium entry (SOCE) occurring after the depletion of the amount of calcium stored in the sarcoplasmic reticulum in mouse differentiated skeletal muscle cells that express wild-type TRIM32 or NHL-Del.

### DETAILED DESCRIPTION OF EXEMPLARY EMBODIMENTS

Hereinafter, the configuration of the present invention will be described in detail.

The “tripartite motif-containing protein 32 (TRIM32)” used in the present invention is a constituent for E3 ubiquitin ligase that is involved in the ligation of actin, tropomyosin, and troponin; α-actinin; and a thin filament (also called an actin filament) such as desmin. The TRIM32 is a protein that consists of an N-terminal RING domain, a B-box domain, a coil-coil area, and C-terminal NHL repeats, and originates from a mammal, preferably a human, a mouse, a house mouse, a rabbit, an orangutan, a monkey, a hamster, a cat, a dolphin, or a gorilla. For example, the amino acid sequence of the TRIM32 protein is accessed in GenBank accession no. NP_001093149.1, and its gene sequence is accessed in GenBank accession no. NM_001099679.2.

“NHL repeats” used in the present invention refers to amino acid sequences that are found in a variety of numerous eukaryotic and prokaryotic proteins, have been named after ncl-1, HT2A, and lin-41 for a long time. Particularly, the NHL repeats of the present invention are NHL repeats present in TRIM 32. The NHL repeats may include the amino acid sequence of SEQ ID NO: 1. In addition, polynucleotides encoding the NHL repeats may include the base sequence of SEQ ID NO: 2.

“NHL-Del” used in the present invention refers to a mutant of TRIM32, which means a mutant without NHL repeats of TRIM32. The NHL-Del protein may include the amino acid sequence of SEQ ID NO: 3. In addition, a polynucleotide encoding the NHL-Del may include the base sequence of SEQ ID NO: 4.

“Sarcoplasmic/endoplasmic reticulum Ca²⁺-ATPase 1a (SERCA1a)” used in the present invention refers to a type of Ca²⁺-pump protein that consumes ATP to store calcium in the sarcoplasmic reticulum (SR). This is the key process by which skeletal muscles return to their normal relaxed state after contraction.

To start contraction and maintain it, the skeletal muscles require a large quantity of calcium (on the level of several micromoles) in the cytosol of a skeletal muscle cell, and such calcium is mostly provided by releasing the calcium stored in the sarcoplasmic reticulum into the cytosol through ryanodine receptor 1 (RyR1), which is an internal calcium channel. After contraction, for skeletal muscle relaxation, a process by which the large quantity of calcium supplied to the cytosol for contraction returns into the sarcoplasmic reticulum is required. Here, SERCA1a (1a is the main type of skeletal muscle SERCA), acting as a path for calcium to return to the sarcoplasmic reticulum, consumes ATP and uses that energy to uptake cytosolic calcium into the sarcoplasmic reticulum. Therefore, the role of SERCA1a is very important not only for skeletal muscle relaxation but also for subsequent contraction, and SERCA1a is the key protein for the contraction and relaxation of skeletal muscles. “Skeletal muscle disease” used in the present invention refers to a skeletal muscle disease in which store-operated Ca²⁺-entry (SOCE) is excessively increased or decreased.

“Store-operated Ca²⁺-entry (SOCE)” used in the present invention refers to the entry of external calcium ions into the cytosol through a calcium channel on the cell membrane in response to the phenomenon of the depletion of calcium in the sarcoplasmic reticulum by releasing calcium ions from an intracellular reservoir, that is, the sarcoplasmic reticulum or the endoplasmic reticulum into the cytosol as a signal. Here, STIM1 protein acts as a calcium sensor in the reservoir, and a calcium channel acting as the path for the entry of external calcium in the cell membrane is known as Orai1.

In other words, when SOCE excessively increases, external calcium ions excessively enter the cytosol and are present excessively in the cytosol, and in this case, skeletal muscles are excessively contracted. Accordingly, when SOCE excessively increases, a disease accompanied by excessive skeletal muscle contraction (the continuous excessive contraction of skeletal muscles will ultimately cause problems with both the contraction and relaxation of skeletal muscles in the future) may occur. In the present invention, the skeletal muscle disease with excessively increased SOCE or decreases may be accompanied by muscular cramps or myalgia.

In one embodiment of the present invention, the skeletal muscle disease with excessively increased SOCE may be Stormorken syndrome, Duchenne muscular dystrophy, tubular aggregate myopathy, York platelet syndrome, malignant hyperthermia, or Becker muscular dystrophy, but the present invention is not limited thereto.

In addition, when reducing SOCE, even though calcium ions in the sarcoplasmic reticulum are depleted, a sufficient amount of calcium ions does not enter from the outside. In this case, calcium stored in the sarcoplasmic reticulum cannot be released into the cytosol through RyR1, and an insufficient amount of cytosolic calcium results in weakened contraction of skeletal muscles. Generally, reduced SOCE may appear in the form of muscular hypotonia due to weakening of skeletal muscles caused by type 2H limb-girdle muscular dystrophy (LGMD2H), an elderly skeletal muscle disease, an age-associated skeletal muscle disease, or long-term treatment such as anti-cancer treatment.

“Muscular hypotonia” used in the present invention refers to a condition of reduced muscle resistance. Muscular hypotonia patients often appear with limp arms and legs, have difficulty holding their head, and have difficulty moving, posing, breathing, and speaking. Particularly, muscular hypotonia occurs in normal elderly people with reduced muscle strength and also appears in the form of a secondary disease with cancer, AIDS, and geriatric illnesses.

In one embodiment of the present invention, the skeletal muscle disease in which SOCE is excessively reduced may be LGMD2H, sarcopenia, dynapenia, or congenital non-progressive muscular hypotonia, but the present invention is not limited thereto.

The inventors discovered that binding of TRIM32 to SERCA1a increases the level of cytosolic calcium for skeletal muscle contraction, and the internal calcium migration for skeletal muscle contraction. The interaction between TRIM32 and SERCA1a is performed by the NHL repeats of TRIM32. Therefore, a mutant of TRIM32, preferably a mutant of the NHL repeats in TRIM32, such as the NHL-Del, may not interact with SERCA1a.

Accordingly, it can be used for screening or diagnosis of a drug for the treatment of a skeletal muscle disease by measuring the interaction of TRIM32 or NHL repeats with SERCA1a.

The present invention provides a composition for diagnosing a skeletal muscle disease, which includes an agent for measuring the interaction(s) of TRIM32 and/or NHL repeats with SERCA1a.

The term “diagnosis” used in the present invention refers to a series of actions to confirm the presence or characteristic of a disease in a subject. The diagnosis in the present invention may also include an action to determine the occurrence of a disease. In addition, the diagnosis in the present invention may include an action to determine the risk of developing a disease.

“Subject” used in the present invention refers to an object for which the risk of developing a disease is to be confirmed, and more particularly, can be any mammal, including, for example, not only a human or other primate, but also livestock including cattle, pigs, sheep, horses, dogs, and cats without limitation, but is preferably a human.

In one embodiment of the present invention, the measurement of protein interaction or the measurement of the expression level of a protein may be the measurement of the interaction or level of the protein in skeletal muscle cells.

In the present invention, the interaction(s) of TRIM32 and/or NHL repeats with SERCA1a may be confirmed by applying techniques that are conventionally used in the art to confirm a protein-protein or protein-compound reaction. For example, a screening method using a fluorescence assay, a yeast two-hybrid, search of a phage display peptide clone binding to TRIM32 and/or NHL repeats, or SERCA1a, immunoprecipitation, co-immunoprecipitation, reverse co-immunoprecipitation, GST-fusion protein pulldown, affinity chromatography, crosslinking, immunohistochemistry, high throughput screening (HTS) using a natural substance and chemical library, drug-hit HTS, or cell-based screening may be used, but the present invention is not limited thereto.

For example, the interaction(s) of TRIM32 and/or NHL repeats with SERCA1a may be detected using antibodies specific to TRIM32 and/or NHL repeats, or SERCA1a. For example, inhibition may be determined by comparing the amount of the produced antigen-antibody complex with that of the control not treated with a candidate material. The absolute or relative difference in the amount of antigen-antibody complex formation can be confirmed by a molecule-biological or histological analysis method, and such analysis methods include immunoblot, immunoprecipitation, and immunostaining, but the present invention is not limited thereto.

In the detection method using the antigen-antibody reaction, the amount of antigen-antibody complex formation can be measured qualitatively by the signal size of a detection label. “Detection label” used in the present invention refers to a composition that can be detected by a spectroscopic, photochemical, biochemical, immunochemical, chemical, or other physical means. Such detection labels may include enzymes, fluorescent materials, ligands, luminescent materials, microparticles, redox molecules, and radioisotopes, but the present invention is not limited thereto.

As another example, after connecting a reporter protein to TRIM32 and/or NHL repeats, or SERCA1a, the binding of TRIM32 and/or NHL repeats to SERCA1a may be detected by the signal size of the reporter protein.

The reporter protein may be luciferase, chloramphenicol acetyltransferase, β-glucuronidase, β-galactosidase, alkaline phosphatase, or a fluorescent protein. Here, the fluorescent protein may be a green fluorescent protein, red fluorescent protein, blue fluorescent protein, yellow fluorescent protein, cyan fluorescent protein, enhanced green fluorescent protein, enhanced red fluorescent protein, enhanced blue fluorescent protein, enhanced yellow fluorescent protein, or enhanced cyan fluorescent protein. When the reporter protein is a fluorescent protein, it is possible to confirm whether the interaction(s) of TRIM32 and/or NHL repeats with SERCA1a is inhibited by measuring the fluorescence intensity of the reporter protein using a fluorescence microscope, and when a luminescent enzyme such as luciferase is used as a reporter protein, it is possible to confirm whether protein interaction is increased by a known luciferase activity measurement method or luciferase luminescence. In addition, when an enzyme that can convert a substance into a chromogenic product emitting detectable light, such as chloramphenicol acetyltransferase, β-glucuronidase, β-galactosidase, or alkaline phosphatase, is used as a reporter protein, it is possible to confirm whether protein interaction is increased by a light signal obtained by an enzymatic reaction using a chromogenic substrate.

The composition for diagnosing a skeletal muscle disease according to the present invention may be provided in the form of a diagnostic kit. The kit may be manufactured by a conventional method known in the art.

In addition, the kit of the present invention may include an agent for measuring the interaction(s) of TRIM32 and/or NHL repeats with SERCA1a as well as a composition, solution, or device consisting of one or more types of other components suitable for an analysis method.

The present invention provides a method of providing information for the diagnosis of a skeletal muscle disease, which includes measuring the interaction(s) of TRIM32 and/or NHL repeats with SERCA1a from a biological sample isolated from a subject.

All content described above regarding the diagnostic composition and kit may be applied or adapted intactly to the above method.

“Biological sample” used in the present invention encompasses tissue, cells, whole blood, serum, plasma, saliva, sputum, cerebrospinal fluid, or urine obtained from a subject, but the present invention is not limited thereto.

In one embodiment of the present invention, compared with the normal control according to the information providing method, when the interaction(s) of TRIM32 and/or NHL repeats with SERCA1a is increased compared to the normal control, it can be determined that a skeletal muscle disease with increased SOCE occurs, or that the possibility of the occurrence of a skeletal muscle disease is high.

In one embodiment of the present invention, compared with a normal control according to the information providing method, when the interaction(s) of TRIM32 and/or NHL repeats with SERCA1a is decreased compared to the normal control, it can be determined that a skeletal muscle disease with decreased SOCE occurs, or that the possibility of the occurrence of a skeletal muscle disease is high.

The present invention provides a method of screening a drug for treating a skeletal muscle disease.

In the present invention, the candidate material may be a material that is estimated to have potential as a medicine increasing the interaction(s) of TRIM32 and/or NHL repeats with SERCA1a in skeletal muscle cells according to a conventional selection method, or a randomly selected individual nucleic acid, protein, peptide, extract, natural substance, or compound.

All content described above regarding the diagnostic composition and kit, and the method of providing information may be applied or adapted intactly to the above method.

A candidate material that increases the interaction(s) of TRIM32 and/or NHL repeats with SERCA1a in skeletal muscle cells, obtained through the screening method of the present invention, may be a candidate material for a therapeutic agent for a skeletal muscle disease with decreased SOCE. That is, when the candidate material increases the interaction(s) of TRIM32 and/or NHL repeats with SERCA1a in skeletal muscle cells, the candidate material may be determined as a material that can treat a skeletal muscle disease with decreased SOCE.

In addition, a candidate material that decreases the interaction(s) of TRIM32 and/or NHL repeats with SERCA1a in skeletal muscle cells, obtained by the screening method of the present invention, may be a candidate material for a therapeutic agent for a skeletal muscle disease with increased SOCE. In other words, when the candidate material decreases the interaction(s) of TRIM32 and/or NHL repeats with SERCA1a in skeletal muscle cells, the candidate material may be determined as a material that can treat a skeletal muscle disease with increased SOCE.

Such a candidate material for a therapeutic agent for a skeletal muscle disease acts as a leading compound during the subsequent development of a therapeutic agent for a skeletal muscle disease, and the structure of the leading compound may be modified or optimized to exhibit an effect of increasing or decreasing the interaction(s) of TRIM32 and/or NHL repeats with SERCA1a in skeletal muscle cells, thereby developing a novel therapeutic agent for a skeletal muscle disease.

In addition, the present invention provides a pharmaceutical composition for preventing or treating a skeletal muscle disease.

All content described above regarding the diagnostic composition, the information providing method for diagnosis, and the drug screening method may be applied or adapted intactly to the above method.

In one embodiment of the present invention, as a result of expressing NHL-Del in mouse differentiated skeletal muscle cells, the inventors confirmed that SOCE increased by the expression of the normal control or wild-type TRIM32 is decreased.

Accordingly, the present invention provides a pharmaceutical composition for treating a skeletal muscle disease, which includes NHL-Del or a polynucleotide encoding the NHL-Del as an active ingredient.

For the pharmaceutical composition of the present invention, the skeletal muscle disease is a skeletal muscle disease with increased SOCE. In one embodiment of the present invention, the skeletal muscle disease with increased SOCE may be Stormorken syndrome, Duchenne muscular dystrophy, tubular aggregate myopathy, York platelet syndrome, malignant hyperthermia, or Becker muscular dystrophy, but the present invention is not limited thereto.

In addition, the pharmaceutical composition of the present invention may further include a pharmaceutically acceptable carrier. Pharmaceutically acceptable carriers include a carrier and a vehicle, which are conventionally used in the medical field, and particularly, an ion exchange resin, alumina, aluminum stearate, lecithin, a serum protein (e.g., human serum albumin), a buffer (e.g., various types of phosphates, glycine, sorbic acid, potassium sorbate, and a partial glyceride mixture of saturated vegetable fatty acids), water, a salt, an electrolyte (e.g., protamine sulfate, disodium hydrogen phosphate, potassium hydrogen phosphate, sodium chloride, and a zinc salt), colloidal silica, magnesium trisilicate, polyethylene glycol, sodium carboxymethylcellulose, polyacrylate, wax, or wool grease, but the present invention is not limited thereto.

In addition, the composition of the present invention may further include a lubricant, a wetting agent, an emulsifier, a suspending agent, or a preservative, in addition to the above-described components.

In one embodiment, the composition according to the present invention may be prepared as an aqueous solution for parenteral administration, and preferably, Hank's solution, Ringer's solution, or a buffer solution such as physically buffered saline. An aqueous injection suspension may include a substrate that can increase the viscosity of the suspension, such as sodium carboxymethylcellulose, sorbitol, or dextran.

The composition of the present invention may be systemically or locally administered, and may be formulated into a suitable formulation according to a known art for the administration. For example, for oral administration, the composition may be administered by mixing it with an inert diluent or edible carrier, being sealed in a hard or soft gelatin capsule, or being compressed into tablets. For oral administration, the active compound may be mixed with excipients and used in the form of an ingestible tablet, buccal tablet, troche, capsule, elixir, suspension, syrup, or wafer.

Various formulations for injectable and parenteral administration may be prepared according to a method known in the art or a conventional method. In addition, an effective amount of NHL-Del or a polynucleotide encoding the NHL-Del may be administered after being prepared in a solution immediately before the administration to saline or buffer via an appropriate route such as intravenous, subcutaneous, intramuscular, intraperitoneal, or transdermal administration.

“Administration” used in the present invention refers to the introduction of the composition of the present invention into a patient according to an appropriate method, and the administration route of the composition of the present invention may be any of various oral or parenteral routes to reach target tissue. The composition of the present invention may be administered intraperitoneally, intravenously, intramuscularly, subcutaneously, intradermally, orally, topically, intranasally, intrapulmonarily, or intrarectally, but the present invention is not limited thereto.

In addition, the present invention provides a method of treating a skeletal muscle disease, which includes administering a therapeutically effective amount of NHL-Del or a polynucleotide encoding the NHL-Del to a subject in need thereof.

The subject referred to herein is a mammal that is a target for treatment, observation, or experiment, and is preferably a human.

“Effective amount” used herein refers to an amount necessary to delay or completely stop the onset or progression of a target disease to be treated, and the effective amount of NHL-Del or a polynucleotide encoding the NHL-Del included in the pharmaceutical composition of the present invention is an amount required for adjusting SOCE to a normal level in a skeletal muscle disease with increased SOCE. Accordingly, the effective amount may be adjusted according to various factors, including the type of disease, the severity of a disease, the types and contents of other components contained in the composition, and a patient's age, body weight, general health condition, sex, and diet, administration time, an administration route, the duration of treatment, and a current drug. It is obvious to those of skill in the art that an appropriate total daily dose can be determined by a physician within the context of proper medical judgment.

For the purpose of the present invention, it is desirable to apply the specific therapeutically effective amount for a specific patient differently depending on various factors, including the type and degree of response to be achieved, a specific composition that can be used with a different agent in some cases, a patient's age, body weight, general health condition, sex, and diet, administration time, administration route, the excretion rate of the composition, the duration of treatment, and any drugs used concurrently or simultaneously with the specific composition, and similar factors well known in the medical field.

“Treatment” used in the present invention refers to an approach for obtaining a beneficial or preferable clinical outcome. For the purpose of the present invention, beneficial or desirable clinical outcomes include, but are not limited to, symptom relief, reduction in the scope of a disease, the stabilization of a disease state (i.e., not worsening), the delay or decrease in progression of a disease, the improvement or temporary relief and mitigation (partially or wholly) of a disease state, and whether it is detected or not. “Treatment” may also refer to increasing a survival rate compared to an expected survival rate without treatment. “Treatment” refers to both therapeutic treatment and a preventive method or action. The treatments include the treatment required for a disorder that has already occurred, as well as a disorder that is prevented. “Mitigation” of a disease may mean that, compared to when not treated, the scope of a disease state and/or undesirable clinical signs decrease, and/or the time course of progression is delayed or extended.

The advantages and features of the present invention and the methods of accomplishing the same will become apparent with reference to the following examples to be described in detail and the accompanying drawings. However, the present invention is not limited to the exemplary embodiments disclosed below and may be embodied in many different forms. These exemplary embodiments are merely provided to complete the disclosure of the present invention and fully convey the scope of the present invention to those of ordinary skill in the art, and the present invention should be defined by only the accompanying claims.

### Experimental Method

This experiment was conducted in accordance with the regulations and guidelines of the College of Medicine at the Catholic University (Ethics Approval Regulation, 2017-0117-01). Sites where animal work progressed and all surgical interventions complied with the Guidelines and Policies for Rodent Survival Surgery approved by the Institutional Animal Care and Use Committee of the College of Medicine at the Catholic University of Korea, the Laboratory Animals Welfare Act, and the Guide for Care and Use of Laboratory Animals. All protocols for the experiment were approved by the Committee of the College of Medicine at the Catholic University of Korea.

### 1-1. Construction of cDNA for GST-Tagged NHL Repeat Protein (GST-NHL) and Expression Method to Protein

PCR was performed using human TRIM32 cDNA (GenBank accession code: NM_012210.3) as a template and PCR primers listed in Table 1. The obtained PCR product was inserted into pGEX-4T-1, its base sequence was confirmed again through a protein sequencer, and the resulting product was transformed to E. coli (DH5α) to express a protein and purified using GST beads.

TABLE 1
PCR primer sequences for constructing cDNA for
NHL repeat protein (Xho I and BamH I)

| | |
|---|---|
| Forward primer | 5′-CGGAATTCATGTTCAATCTTCC-3′ |
| (SEQ ID NO: 5) | |
| Reverse primer | 5′-CCTCGAGTCATGGGGTGGAATATC-3′ |
| (SEQ ID NO: 6) | |

### 1-2. Preparation of Triad Samples and GST-NHL Binding Reaction

A triad sample was obtained by obtaining a triad vesicle from rabbit skeletal muscle tissue (rabbit fast-twitch skeletal muscle (back and leg)) and solubilizing the triad vesicle using a lysis buffer (1% Triton X-100, 10 mM Tris-HCl, 1 mM Na₃ VO₄, 10% glycerol, 150 mM NaCl, 5 mM EDTA, a protease inhibitor cocktail, and pH 7.4) at 4° C. for 4 hours. For binding analysis, GST-NHL was attached to a GST bead (Abcam, Cambridge, MA; Cat. No. ab193267) and mixed with the triad sample to allow a binding reaction at 4° C. for 8 hours. The proteins attached to the obtained beads were separated from an SDS-PAGE gel and visualized through Coomassie brilliant blue staining.

### 1-3. Protein Identification Through MALDI-TOF/TOF Mass Spectrometry and Database Searching

Proteins obtained through the triad sample preparation and the binding reaction with GST-NHL were digested with trypsin in a gel state. The digested peptide solution was desalted and concentrated using a C18 nano column (100 to 300 nL of POROS reverse phase R2 material with a size of 20 to 30-μm; Thermo Fisher Scientific, Waltham, MA; Cat. No. 1102412), 50% MeOH, 49% H₂O, and 1.5 mL of 1% formic acid. Masses of peptides were assessed by a MALDI-TOF/TOF mass spectrometer (4700 Proteomics Analyzer MALDI-TOF/TOF, Applied Biosystems, Waltham, MA). The assessed results were searched using the NCBI database to identify corresponding proteins.

### 1-4. Co-Immunoprecipitation

Co-immunoprecipitation was performed on triad samples using SERCA1a antibodies. The obtained resultants were separated from a 10% SDS-PAGE gel, and the proteins separated from the gel were transferred to a polyvinylidene fluoride (PVDF) membrane (100 V for 2 hrs) and treated with 5% non-fat milk for 1 hour. Afterward, the resultants were treated with corresponding primary antibodies (3 to 12 hrs, TRIM32 antibodies or SERCA1a antibodies) and then with corresponding secondary antibodies (horseradish peroxidase-conjugated secondary antibodies) for 45 minutes, followed by visualization and analysis through a chromogenic reaction (SuperSignal ultrachemiluminescent substrate). To perform an immunoblot test using mouse differentiated skeletal muscle cells (myotubes), the differentiated skeletal muscle cells were treated with a lysis buffer (consisting of 1% Triton X-100, 10 mM Tris-HCl (pH 7.4), 1 mM Na₃VO₄, 10% glycerol, 150 mM NaCl, 5 mM EDTA, and protease inhibitors (1 μM pepstatin, 1 μM leupeptin, 1 mM phenylmethylsulfonyl fluoride, 20 mg/ml aprotinin, and 1 μM trypsin inhibitor)) to solubilize at 4° C. for 24 hours. Here, as an example, differentiated skeletal muscle cells, which were differentiated on a 10 cm culture plate, were treated with 300 uL of lysis buffer. Subsequent antibody treatments and chromogenic reactions were performed as described above (the co-immunoprecipitation method using triad samples).

### 1-5. Construction of cDNA for NHL-Del and Protein Expression Method

To express NHL-Del in mammal cells, PCR was performed using human TRIM32 cDNA (GenBank accession code: NM_012210.3) as a template and PCR primers listed in Table 2. The obtained PCR product was transferred to a pEGFP-N1 vector, thereby constructing NHL-Del cDNA for mammalian cell expression. The base sequence of the completed cDNA was reconfirmed using a genome base sequencer, and wild-type TRIM32 for mammalian cell expression was obtained from Addgene (Cat #69541).

TABLE 2
PCR primer sequences for constructing cDNA for
NHL-Del (Xho I and BamH I)

| | |
|---|---|
| Forward primer | 5′-GACTCAGATCTCGAGGCCACCATG-3′ |
| (SEQ ID NO: 7) | |
| Reverse primer | 5′-CGGGATCCCATTCCTGGAGTG-3′ |
| (SEQ ID NO: 8) | |

### 1-6. Isolation of Skeletal Muscle Satellite Cells and Differentiation Method to Differentiated Skeletal Muscle Cells (Myotubes)

Skeletal muscle progenitor cells (myoblasts) were obtained by isolating skeletal muscle satellite cells from mouse skeletal muscles and primary culturing the cells. In the culturing process, the cells were cultured in culture media (F10 Nutrient Mixture consisting of 20% FBS, 100 units/ml penicillin, 100 μg/ml streptomycin, 2 mM L-glutamine, and 20 nM basic fibroblast growth factor) in a 5% CO₂ incubator at 37° C. 10 cm or 96-well culture plates were used according to a purpose, and all plates were used after being coated with Matrigel. When the cells were grown until the confluence of approximately 70% of the culture plates, differentiation to differentiated skeletal muscle cells was induced (cell differentiation solution used 5% heat-inactivated horse serum and low-glucose DMEM, instead of 20% FBS and F-10 Nutrient Mixture in the cell culture medium, did not use bFGF, and used an 18% CO₂ incubator). The cells that had completed differentiation were ‘differentiated skeletal muscle cells (myotubes)’ and were used in the following experiment.

### 1-7. Expression of Wild-Type TRIM32 and NHL-Del in Differentiated Skeletal Muscle Cells (Myotubes)

Immature skeletal muscle cells (immature myotubes) on differentiation day 3 were transfected with cDNA for wild-type TRIM32 or NHL-Del for 4 hours (cDNA transfection: each cDNA was formed in a pEGFP-NI vector, and cells that had been cultured in a 10 cm culture plate, for example, were treated with both 30 μL FuGENE6 and 20 μg cDNA). Further differentiation of the cDNA-transfected cells was induced for 36 hours. The cells in this state were differentiated skeletal muscle cells (myotubes) that expressed wild-type TRIM32 and NHL-Del and had completed differentiation, and were used in the following experiment.

### 1-8. Immunocytochemistry

To confirm the expression of wild-type TRIM32 and NHL-Del in differentiated skeletal muscle cells, immunocytochemistry was performed. The differentiated skeletal muscle cells were fixed with cold methanol for 30 minutes and then treated with GFP primary antibodies (1:500) and cy3-conjugated secondary antibodies (1:500, Sigma).

### 1-9. Method of Measuring Width of Differentiated Skeletal Muscle Cells (Myotubes)

The lengths of the thickest parts of the differentiation-completed differentiated skeletal muscle cells were Measured using the program ImageJ.

### 1-10. Immunoblot analysis

Samples were separated from an 8%, 10% or 12% SDS-PAGE gel, proteins separated from the gel were transferred to a PVDF membrane (100 V for 2 hrs) and treated with 5% non-fat milk for 1 hour. Afterward, the resultants were treated with corresponding primary antibodies (1:1000), and then with corresponding secondary antibodies (horseradish peroxidase-conjugated secondary antibodies) for 45 minutes, followed by visualization and analysis through a chromogenic reaction (SuperSignal ultrachemiluminescent substrate).

### 1-11. Method of Measuring Calcium Reaction of Differentiated Skeletal Muscle Cells (Myotubes) to Caffeine or KCl by Single Cell Calcium Imaging Technique

The calcium fluorescent dye (Ca²⁺ dye), fluo-4 (5 μM), which, when bonded with calcium, emits fluorescence at a different wavelength than before the calcium was bonded, was added to differentiated skeletal muscle cells and the cells were incubated at 37° C. for 45 minutes. Here, the differentiated skeletal muscle cells had been treated with an imaging solution (125 mM NaCl, 5 mM KCl, 2 mM KH₂PO₄, 2 mM CaCl₂, 25 mM HEPES, 6 mM glucose, 1.2 mM MgSO4, 0.05% BSA (fraction V), and pH 7.4). To measure calcium migration in the cells, a fluorescence microscope was used (Nikon x40 oil-immersion objective, NA 1.30, ECLIPSE Ti, Nikon). The fluorescence change of the calcium fluorescent dye was transferred to a computer using a 75-watt Xenon lamp (FSM150Xe, Bentham Instruments, Ltd) and a 12-bit CCD camera (DVC-340M-OO-CL, Digital Video Camera Company), which were connected to the fluorescence microscope, and analyzed using InCyt Im1 image acquisition and analysis software (v5.29, Intracellular Imaging Inc). The calcium migration from the sarcoplasmic reticulum (SR) to the cytosol by caffeine or KCl treatment and cytosolic calcium concentration were measured, and values for the peak heights of the graph were statistically processed (showing the same tendency as the statistical processing of values for the areas of the graph).

### 1-12. Method of Measuring External Calcium Entry (SOCE) by Single Differentiated Skeletal Muscle Cell (Myotube) Calcium Imaging Technique

The calcium fluorescent dye (Ca²⁺ dye), fluo-4 (5 μM), which, when bonded with calcium, emits fluorescence at a different wavelength than before the calcium is bonded, was added to differentiated skeletal muscle cells and the cells were incubated at 37° C. for 45 minutes. Here, the differentiated skeletal muscle cells had been treated with an imaging solution (125 mM NaCl, 5 mM KCl, 2 mM KH₂PO₄, 2 mM CaCl₂, 25 mM HEPES, 6 mM glucose, 1.2 mM MgSO₄, 0.05% BSA (fraction V), and pH 7.4). To measure calcium migration in the differentiated skeletal muscle cells, a fluorescence microscope (Nikon x40 oil-immersion objective, NA 1.30, ECLIPSE Ti, Nikon) was used. The fluorescence change of the calcium fluorescent dye was transferred to a computer using a 75-watt Xenon lamp (FSM150Xe, Bentham Instruments, Ltd) and a 12-bit CCD camera (DVC-340M-OO-CL, Digital Video

Camera Company), which were connected to the fluorescence microscope, and analyzed using InCyt Im1 image acquisition and analysis software (v5.29, Intracellular Imaging Inc). When calcium in the calcium reservoir (i.e., the sarcoplasmic reticulum (SR)) was depleted, for the experiment of measuring the amount of calcium (store-operated Ca²⁺ entry (SOCE)) entering from the outside to the inside of the cells, the differentiated skeletal muscle cells were treated with a Ca²⁺-free imaging solution which did not have calcium for 5 minutes, and then with thapsigargin (TG) to induce calcium depletion in the sarcoplasmic reticulum. The amount of calcium entering from the outside of the cells was measured by the treatment of 2 mM calcium to the outside of the cells. The cells were treated with TG that had been dissolved in Me₂SO (<0.05%) in accordance with the manual, confirming that there was no change in calcium migration in the cells due to Me₂SO (<0.05%). The results of the measurement of the reaction of SOCE and TG were analyzed by statistically processing the areas of the calcium migration graph.

### 1-13. Data Analysis (Statistical Analysis)

Data obtained through numerous experiments was collected and expressed as means ±SE. A value obtained from the control was set as 1 and shown as a normalized ratio expressing a relative change thereto. Significant differences were evaluated using an unpaired t-test or one-way ANOVA-Tukey's hoc test (GraphPad InStat, v2.04) and marked (* or #) at P<0.05. Graphs were plotted using the program Origin 2019b.

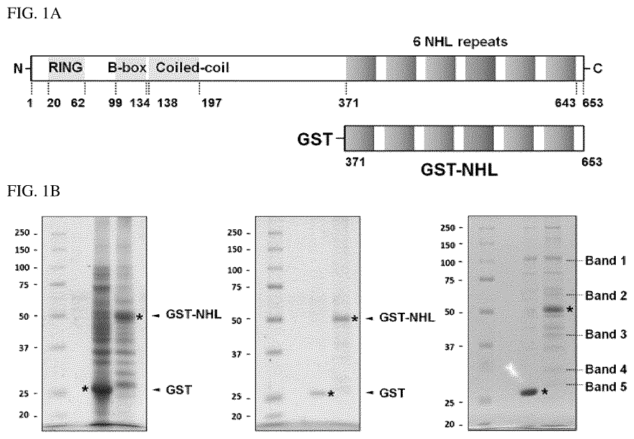

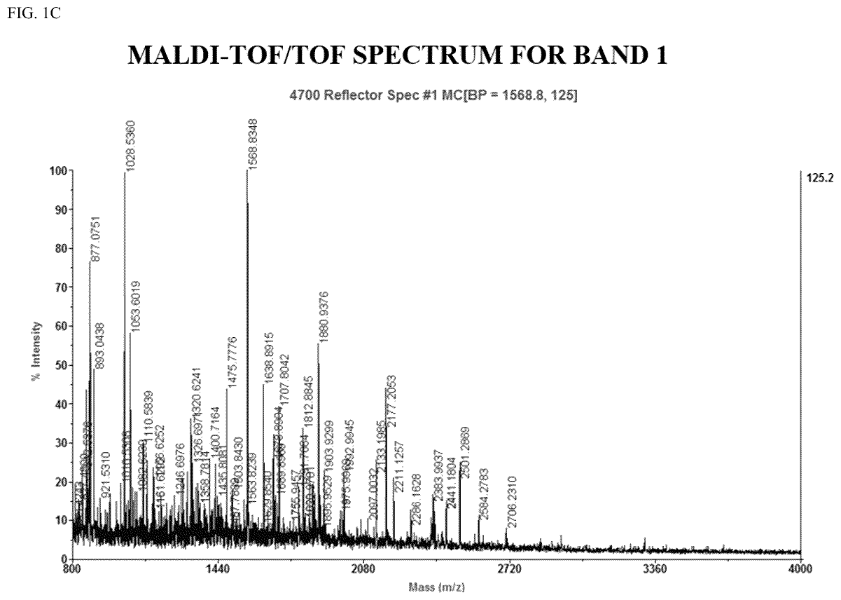

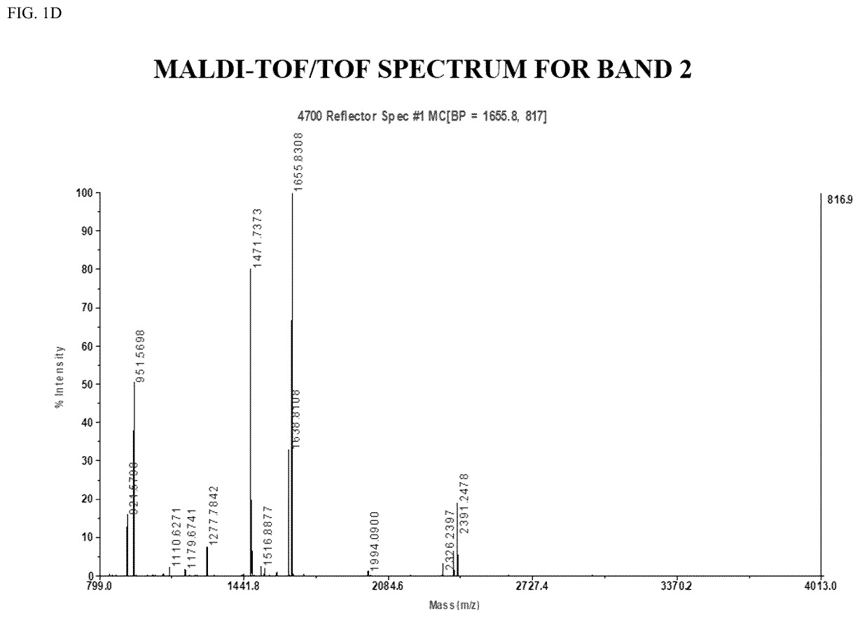

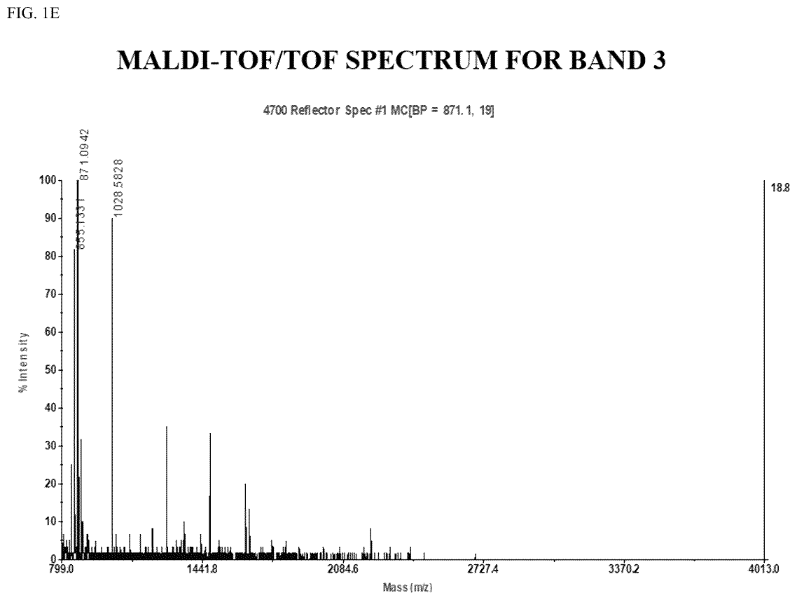

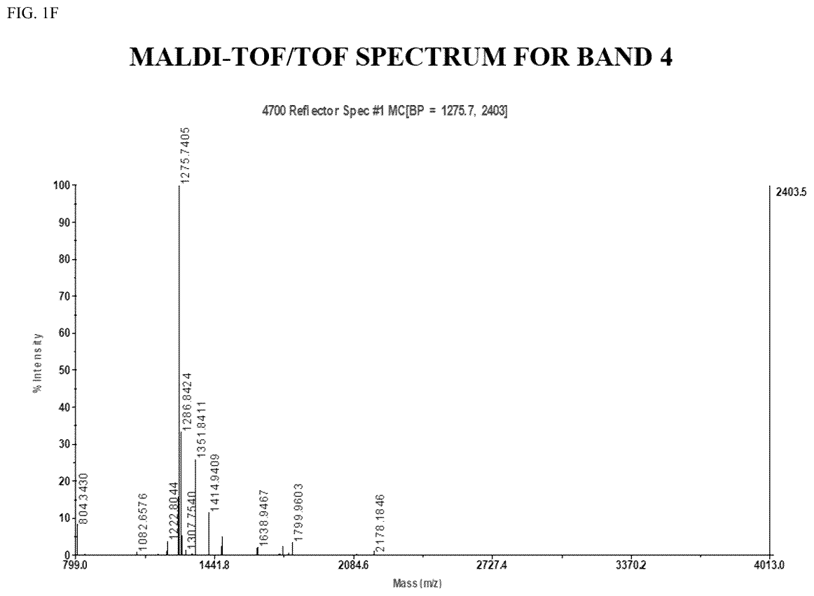

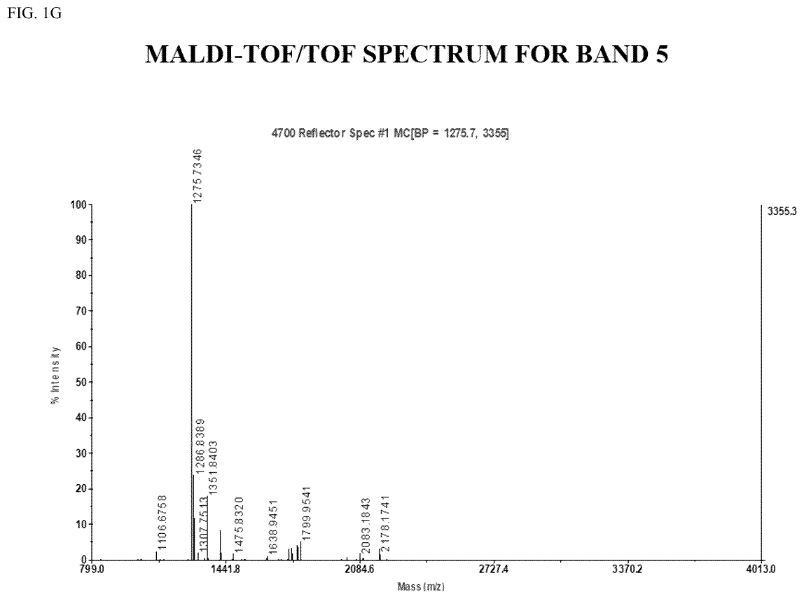

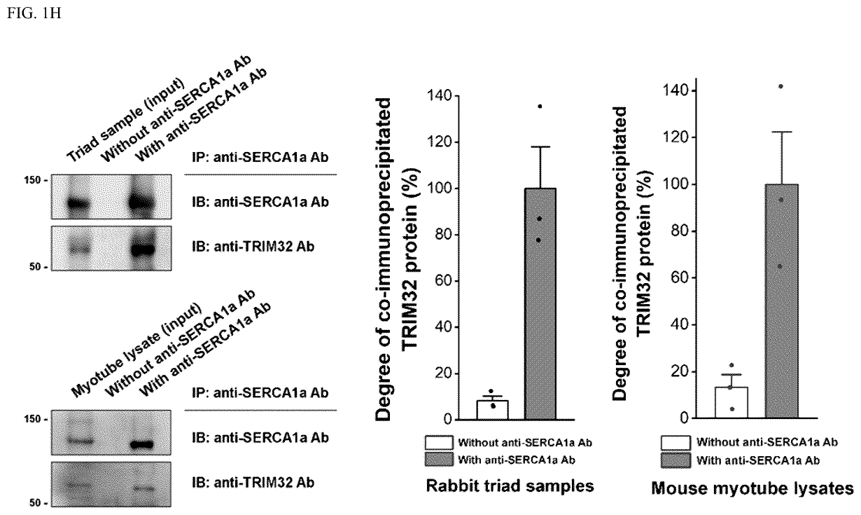

### Example 1. Confirmation of Reaction of Bonding TRIM32 and NHL Repeats with SERCA1a (FIG. 1)

To find a protein that binds to the NHL repeats of TRIM32 in skeletal muscle, NHL repeats were constructed as N-terminal GST tagged form (GST-NHL) and expressed in E. coli (FIG. 1A). The successful expression of the GST-NHL protein expressed by being transformed to E. coli (DH5α) was confirmed by Coomassie blue staining (left in FIG. 1B). GST-NHL was attached to GST beads and purified, the purified degree of GST-NHL was confirmed by Coomassie blue staining (middle in FIG. 1B), and the reaction of bonding a triad sample with GST-NHL obtained in rabbit skeletal muscle was confirmed by Coomassie blue staining (right in FIG. 1B). Here, GST was used as a negative control. Five triad proteins that appeared to bind to GST-NHL were represented with bands 1 to 5. The identification of each of the five proteins was carried out through MALDI-TOF/TOF mass spectrometry (FIGS. 1C to 1G) and database (Table 3), and band 1 was determined as SERCA1a.

TABLE 3

| Band | | GenBank | | Matching |
|---|---|---|---|---|
| # | Protein name | accession code | Species | score |
| 1 | Sarcoplasmic/ | NM_001089318 | Rabbit | 103 |
| | endoplasmic | | | |
| | reticulum calcium | | | |
| | ATPase 1a (SERCA1a) | | | |
| 2 | TRIM32 | XP_002720557.1 | Rabbit | 180 |
| 3 | No matched signal | | | |
| 4 | TEM-1 Beta-lactamase | ACU00830.1 | E. Coli | 276 |
| 5 | TEM-1b Beta-lactamase | ADB90406.1 | E. Coli | 375 |

Co-immunoprecipitation was performed using the triad samples obtained from rabbit skeletal muscle and SERCA1a antibody, and immunoblot analysis was performed on the obtained samples using SERCA1a and TRIM32 antibodies (top in FIG. 1H). Co-immunoprecipitation was performed using a lysate sample of mouse differentiated skeletal muscle cells and SERCA1a antibody, and immunoblot analysis was performed on the obtained sample using SERCA1a and TRIM32 antibodies (bottom in FIG. 1H). The value obtained by adding the antibody (without anti-SRECA1a Ab) was set to 1, and the relative change thereto was calculated as a normalized ratio and is shown in the bar graph on the right in FIG. 1H. IB, IP, and Ab represent immunoblot, co-immunoprecipitation, and antibody, respectively, and * indicates a significant difference compared to ‘Without anti-SRECAla Ab’ (p <0.05).

As a result, TRIM32 binding to SERCA1a via NHL repeats was found in both rabbit skeletal muscle tissue and mouse skeletal muscle cells, revealing that binding of TRIM32 to SERCA1a via NHL repeats is a common phenomenon.

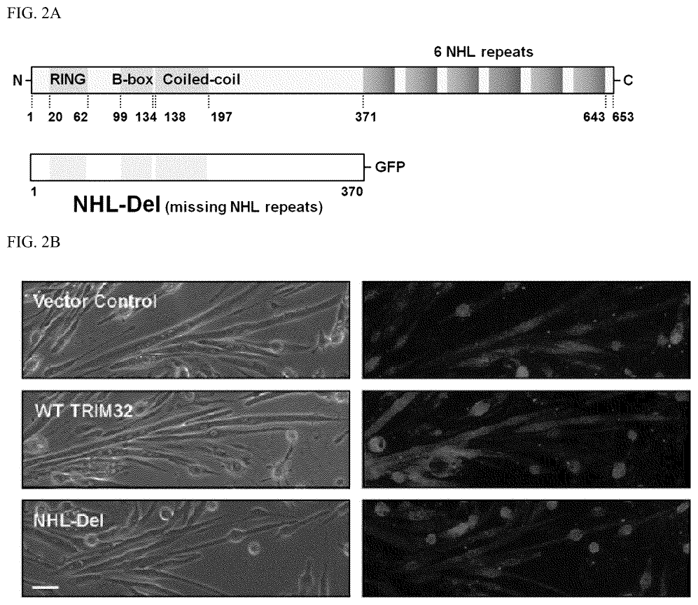

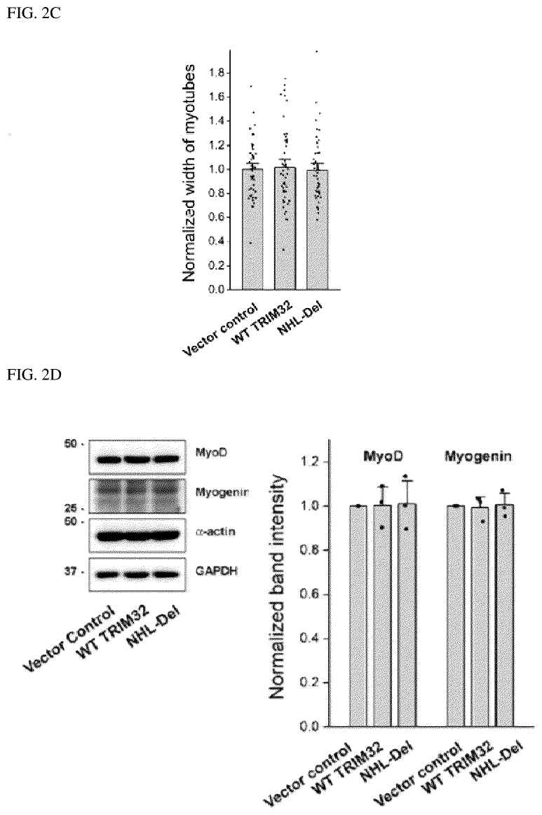

### Example 2. Comparison of Wild-Type TRIM32 or NHL-Del Expression in Mouse Differentiated Skeletal Muscle Cells and Differentiation States (FIG. 2)

The expression of a vector (vector control), wild-type TRIM32, or NHL-Del in differentiated skeletal muscle cells was confirmed by immunochemistry (FIGS. 2A and 2B). The characteristics of the mouse differentiated skeletal muscle cells expressing wild-type TRIM32 or NHL-Del are shown in Table 4. The widths of the thickest parts of the differentiated skeletal muscle cells were measured and compared with those of the control, and the number of experiments used in the analysis and statistics and the values obtained thereby are shown in parentheses in Table 4.

TABLE 4

| | Vector control | WT TRIM32 | NHL-Del |
|---|---|---|---|
| Width of myotubes | 1.00 ± 0.03 | 1.01 ± 0.04 | 0.99 ± 0.04 |
| | (50 myotubes) | (50 myotubes) | (50 myotubes) |
| Releasable Ca²⁺ | 1.00 ± 0.15 | 1.48 ± 0.18 * | 0.99 ± 0.15 |
| level from SR | (72 myotubes | (82 myotubes | (81 myotubes |
| | from 25 wells) | from 18 wells) | from 22 wells) |
| Caffeine response | 1.00 ± 0.07 | 1.22 ± 0.09 * | 0.97 ± 0.07 |
| | (85 myotubes | (58 myotubes | (63 myotubes |
| | from 63 wells) | from 50 wells) | from 61 wells) |
| KCl response | 1.00 ± 0.06 | 1.27 ± 0.11 * | 0.94 ± 0.06 |
| | (85 myotubes | (56 myotubes) | (61 myotubes |
| | from 52 wells) | from 36 wells) | from 47 wells) |

The differentiation marker proteins (MyoD and myogenin proteins) showing the degree of differentiation of the differentiated skeletal muscle cells were confirmed by immunoblot analysis, and the degree of the expression of the housekeeping protein, α-actin, in all cells was confirmed (no change). GAPDH protein was used as a loading control, the value of the control was set to 1, and the relative changes thereto were calculated as normalized ratios and are shown in bar graphs (FIGS. 2C and 2D). Three independent experiments per protein were performed, and the values were expressed as the mean ±SE obtained from the three independent experiments. Expression levels of the proteins were normalized to the average value of the vector control. The number of experiments used in the analysis and statistics and the values obtained thereby are shown in parentheses in Table 5. * indicates a significant difference compared to the control (p<0.05).

TABLE 5

| | Vector control | WT TRIM32 | NHL-Del |
|---|---|---|---|
| MyoD | 1.00 ± 0.00 | 1.00 ± 0.06 | 1.01 ± 0.07 |
| Myogenin | 1.00 ± 0.00 | 0.99 ± 0.03 | 1.01 ± 0.03 |
| RyR1 | 1.00 ± 0.00 | 0.98 ± 0.05 | 1.03 ± 0.05 |
| DHPR | 1.00 ± 0.00 | 1.01 ± 0.04 | 1.02 ± 0.03 |
| SERCA1a | 1.00 ± 0.00 | 0.97 ± 0.03 | 0.98 ± 0.03 |
| TRIM32 | 1.00 ± 0.00 | 1.01 ± 0.06 | 0.99 ± 0.04 |
| CASQ1 | 1.00 ± 0.00 | 1.02 ± 0.02 | 0.99 ± 0.02 |
| MG53 | 1.00 ± 0.00 | 1.02 ± 0.03 | 1.02 ± 0.04 |
| α-actin | 1.00 ± 0.00 | 1.02 ± 0.06 | 1.01 ± 0.02 |

As a result, it was confirmed that wild-type TRIM32 or NHL-Del was successfully expressed in mouse differentiated skeletal muscle cells, and these expressions did not affect the degree of expression in the differentiated skeletal muscle cells.

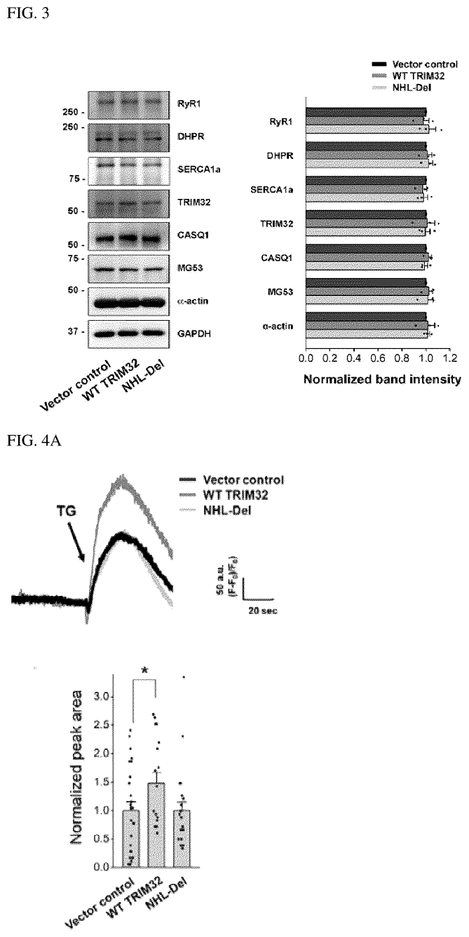

### Example 3. Comparison in Expression Levels of Main Proteins Mediating Skeletal Muscle Contraction and Relaxation in Mouse Differentiated Skeletal Muscle cells that express wild-type TRIM32 or NHL-Del (FIG. 3)

Immunoblot analysis was performed to compare the expression levels of key proteins, such as RyR1, DHPR, SERCA1a, TRIM32 (endogenous), MG53, and CASQ1, which mediate the contraction and relaxation of skeletal muscles in the mouse differentiated skeletal muscle cells that express wild-type TRIM32 or NHL-Del. The degree of the expression of the housekeeping protein in all cells, α-actin, was confirmed (no change). GAPDH protein was used as a loading control, the value of the control was set to 1, and the relative change thereto was expressed as normalized ratios. Three independent experiments per protein were performed, and the values were expressed as the mean ±SE. Expression levels of the proteins were normalized to the average value of the vector control. The number of experiments used in the analysis and statistics and the values obtained thereby are shown in parentheses in Table 5. * indicates a significant difference compared to the control (p<0.05).

As a result, it was confirmed that the expression of wild-type TRIM32 or NHL-Del in mouse differentiated skeletal muscle cells did not affect the expression levels of the key proteins mediating the contraction and relaxation of skeletal muscles. This means that the phenomenon discovered through this study is a phenomenon caused by the expression of TRIM32 or NHL-Del.

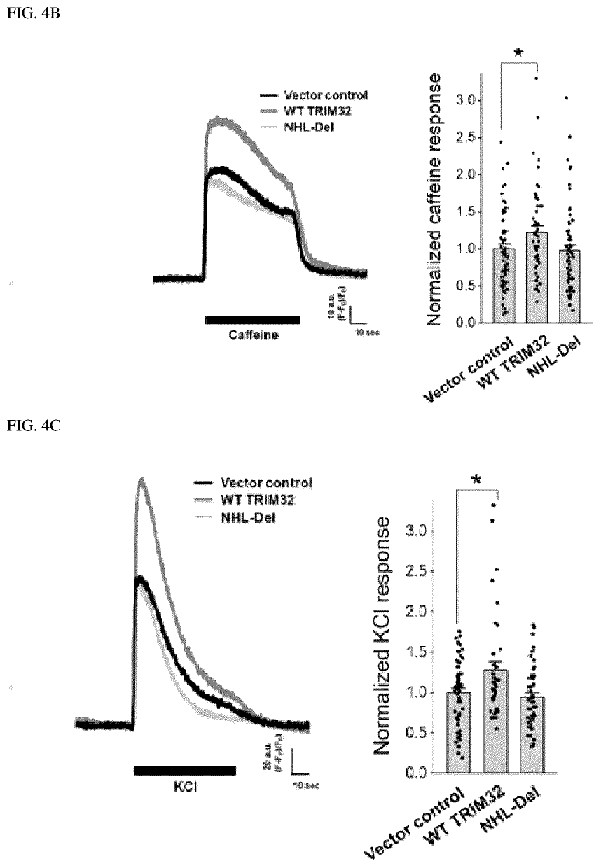

### Example 4. Comparison of Calcium Amount in Sarcoplasmic Reticulum and the Degree of Calcium Migration for Skeletal Muscle Contraction in Mouse Differentiated Skeletal Muscle Cells Expressing Wild-Type TRIM32 or NHL-Del (FIG. 4)

The relative amounts of calcium stored in the sarcoplasmic reticulum were measured and comparison by thapsigargin (TG) in mouse differentiated skeletal muscle cells that express wild-type TRIM32 or NHL-Del was performed (FIG. 4A). In addition, the relative degree of calcium release from the sarcoplasmic reticulum to the cytosol through RyRI for skeletal muscle contraction was measured by treatment of caffeine, which is a direct/specific activator of the internal calcium channel, ryanodine receptor 1 (RyR1), in mouse differentiated skeletal muscle cells that express wild-type TRIM32 or NHL-Del (FIG. 4B). Afterward, the relative amount of released calcium was measured by inducing calcium release from the sarcoplasmic reticulum to the cytosol for skeletal muscle contraction by treatment of KCl, which is a cell membrane depolarizer, in mouse differentiated skeletal muscle cells that express wild-type TRIM32 or NHL-Del (FIG. 4C). * indicates a significant difference compared to the control (p<0.05). The number of experiments used in the analysis and statistics and the values obtained thereby are shown in parentheses in Table 4 of Example 2.

As a result, it was confirmed that wild-type TRIM32 increases a calcium amount in the sarcoplasmic reticulum, and the reaction to caffeine and KCl (that is, representing the degree of calcium migration for contraction during skeletal muscle contraction). However, these phenomena disappear when NHL repeats are eliminated (i.e., NHL-Del with no binding to SERCA1a due to the absence of an SERCA1a-binding site). Therefore, it can be seen that TRIM32 mediates increases in calcium amount in the sarcoplasmic reticulum for skeletal muscle contraction and calcium migration for contraction during skeletal muscle contraction by binding to SERCA1a via its own NHL repeats.

Therefore, it was confirmed that, by the expression of wild-type TRIM32 or NHL repeats, patients with weakened muscle strength or contractility of skeletal muscle caused by LGMD2H, an elderly skeletal muscle disease, an age-associated skeletal muscle disease, or long-term treatment such as anti-cancer treatment, accompanied by muscular hypotonia, can be treated. On the other hand, it was confirmed that skeletal muscle diseases showing excessive skeletal muscle contraction can be treated by NHL-Del expression or antibody treatment to NHL repeats.

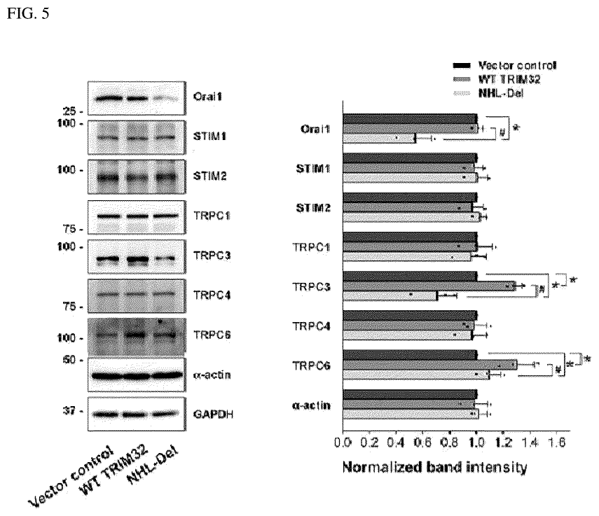

### Example 5. Comparison of Degree of External Calcium Entry (SOCE) Mediating Supply of External Calcium During Skeletal Muscle Contraction in Mouse Differentiated Skeletal Muscle Cells Expressing Wild-Type TRIM32 or NHL-Del (FIG. 5)

The amount of calcium stored in the sarcoplasmic reticulum was depleted by thapsigargin (TG) treatment in mouse differentiated skeletal muscle cells that express wild-type TRIM32 or NHL-Del, and then external calcium entry (SOCE) caused thereby was measured.

As a result, it was confirmed that wild-type TRIM32 increases external calcium entry (i.e., SOCE) mediating calcium supply during skeletal muscle contraction. However, these phenomena disappear when NHL repeats are eliminated (i.e., NHL-Del without NHL repeats, which are SERCA1a-binding sites). Therefore, it can be seen that an increase in calcium supply during skeletal muscle contraction occurs due to the NHL repeats of TRIM32. In addition, it can be identified that NHL-Del without NHL repeats decreases SOCE compared to the control. Accordingly, it was confirmed that, by the expression of wild-type TRIM32 or NHL repeats, patients with weakened muscle strength or contractility of skeletal muscle caused by LGMD2H, an elderly skeletal muscle disease, an age-associated skeletal muscle disease, and long-term treatment, such as anti-cancer treatment, showing the lack of SOCE, can be treated. On the other hand, it was confirmed that skeletal muscle diseases showing excessive skeletal muscle contraction can be treated by NHL-Del expression or antibody treatment to NHL repeats.

According to the present invention, information for diagnosing a skeletal muscle disease can be provided, a skeletal muscle disease can be prevented or treated, and a drug for this can be screened. In addition, the present invention can be useful for prevention or treatment of a skeletal muscle disease.

The present invention has been described with reference to the above-described examples and experimental examples, but this is merely illustrative, and it should be understood by those of ordinary skill in the art that various modifications and equivalent examples and experimental examples can be made therefrom. Therefore, the genuine scope of the present invention should be determined from the technical idea of the accompanying claims.

What is claimed is:

1. A method of screening a drug for the treatment of a skeletal muscle disease, comprising:
treating a biological sample isolated from a subject suspected of having a skeletal muscle disease with a candidate material, and confirming whether the candidate material increases or decreases the interaction(s) of TRIM32 and/or NHL repeats with SERCA1a in skeletal muscle cells.

2. The method of claim 1, wherein the skeletal muscle disease is one or more selected from Stormorken syndrome, Duchenne muscular dystrophy, tubular aggregate myopathy, York platelet syndrome, malignant hyperthermia, Becker muscular dystrophy, type 2H limb-girdle muscular dystrophy (LGMD2H), sarcopenia, dynapenia, and congenital non-progressive muscular hypotonia.

3. The method of claim 1, wherein, when the candidate material decreases the interaction(s) of TRIM32 and/or NHL repeats with SERCA1a in skeletal muscle cells,
it is determined as a therapeutic agent for Stormorken syndrome, Duchenne muscular dystrophy, tubular aggregate myopathy, York platelet syndrome, malignant hyperthermia, or Becker muscular dystrophy.

4. The method of claim 1, wherein, when the candidate material increases the interaction(s) of TRIM32 and/or NHL repeats with SERCA1a in skeletal muscle cells,
it is determined as a therapeutic agent for type 2H limb-girdle muscular dystrophy (LGMD2H), sarcopenia, dynapenia, or congenital non-progressive muscular hypotonia.

5. A method of providing information for the diagnosis of a skeletal muscle disease, comprising:
measuring the interaction(s) of TRIM32 and/or NHL repeats with SERCA1a from a biological sample isolated from a subject.

6. The method of claim 5, wherein the skeletal muscle disease is one or more selected from the group consisting of Stormorken syndrome, Duchenne muscular dystrophy, tubular aggregate myopathy, York platelet syndrome, malignant hyperthermia, Becker muscular dystrophy, type 2H limb-girdle muscular dystrophy (LGMD2H), sarcopenia, dynapenia, and congenital non-progressive muscular hypotonia.

7. The method of claim 5, further comprising, when the interaction(s) of TRIM32 and/or NHL repeats with SERCA1a is increased compared to the normal control,
determining that Stormorken syndrome, Duchenne muscular dystrophy, tubular aggregate myopathy, York platelet syndrome, malignant hyperthermia, or Becker muscular dystrophy occurs or is highly likely to occur.

8. The method of claim 5, further comprising, when the interaction(s) of TRIM32 and/or NHL repeats with SERCA1a is decreased compared to the normal control,
determining that type 2H limb-girdle muscular dystrophy (LGMD2H), sarcopenia, dynapenia, or congenital non-progressive muscular hypotonia occurs or is highly likely to occur.

9. A method of treating a skeletal muscle disease, which includes administering a therapeutically effective amount of NHL-Del or a polynucleotide encoding the NHL-Del to a subject in need thereof,
wherein the disease is Stormorken syndrome, Duchenne muscular dystrophy, tubular aggregate myopathy, York platelet syndrome, malignant hyperthermia, or Becker muscular dystrophy.

10. The method of claim 9, wherein the NHL-Del consists of the amino acid sequence of SEQ ID NO: 3

11. The method of claim 9, wherein the polynucleotide encoding the NHL-Del consists of the base sequence of SEQ ID NO: 4.


SEQUENCE LISTING (8 sequences)

SEQ ID NO: 1 - protein, 283 aa, unidentified
GAKGSTPGMFNLPVSLYVTSQGEVLVADRGNYRIQVFTRKGFLKEIRRSPSGIDSFVLSFLGADLPNLTPLSVAMNCQGLIGVTDSYDNSLKVYTLDGHCVACHRSQLSKPWGITALPSGQFVVTDVEGGKLWCFTVDRGSGVVKYSCLCSAVRPKFVTCDAEGTVYFTQGLGLNLENRQNEHHLEGGFSIGSVGPDGQLGRQISHFFSENEDFRCIAGMCVDARGDLIVADSSRKEILHFPKGGGYSVLIREGLTCPVGIALTPKGQLLVLDCWDHCIKIYS

SEQ ID NO: 2 - DNA, 849 bp, unidentified
  source 1..849: mol_type = genomic DNA
ggggccaaaggcagcactccaggaatgttcaatcttccagtcagtctctacgtgaccagtcaaggtgaagtactagtcgctgaccgtggtaactatcgtatacaagtctttacccgcaaaggctttttgaaggaaatccgccgcagccccagtggcattgatagctttgtgctaagcttccttggggcagatctacccaacctcactcctctctcagtggcaatgaactgccaggggctgattggtgtgactgacagctatgataactccctcaaggtatataccttggatggccactgcgtggcctgtcacaggagccagctgagcaaaccatggggtatcacagccttgccatctggccagtttgtagtaaccgatgtggaaggtggaaagctttggtgtttcacagttgatcgaggatcaggggtggtcaaatacagctgcctatgtagtgctgtgcggcccaaatttgtcacctgtgatgctgagggcaccgtctacttcacccagggcttaggcctcaatctggagaatcggcagaatgagcaccacctggagggtggcttttccattggctctgtaggccctgatgggcagctgggtcgccagattagccacttcttctcggagaatgaggatttccgctgcattgctggcatgtgtgtggatgctcgtggtgatctcatcgtggctgacagtagtcgcaaggaaattctccattttcctaagggtgggggctatagtgtccttattcgagagggacttacctgtccggtgggcatagccctaactcctaaggggcagctgctggtcttggactgttgggatcattgcatcaagatctacagc

SEQ ID NO: 3 - protein, 361 aa, unidentified
MAAAAASHLNLDALREVLECPICMESFTEEQLRPKLLHCGHTICRQCLEKLLASSINGVRCPFCSKITRITSLTQLTDNLTVLKIIDTAGLSEAVGLLMCRSCGRRLPRQFCRSCGLVLCEPCREADHQPPGHCTLPVKEAAEERRRDFGEKLTRLRELMGELQRRKAALEGVSKDLQARYKAVLQEYGHEERRVQDELARSRKFFTGSLAEVEKSNSQVVEEQSYLLNIAEVQAVSRCDYFLAKIKQADVALLEETADEEEPELTASLPRELTLQDVELLKVGHVGPLQIGQAVKKPRTVNVEDSWAMEATASAASTSVTFREMDMSPEEVVASPRASPAKQRGPEAASNIQQCLFLKKM

SEQ ID NO: 4 - DNA, 1083 bp, unidentified
  source 1..1083: mol_type = genomic DNA
atggctgcagcagcagcttctcacctgaacctggatgccctccgggaagtgctagaatgccccatctgcatggagtccttcacagaagagcagctgcgtcccaagcttctgcactgtggccataccatctgccgccagtgcctggagaagctattggccagtagcatcaatggtgtccgctgtcccttttgcagcaagattacccgcataaccagcttgacccagctgacagacaatctgacagtgctaaagatcattgatacagctgggctcagcgaggctgtggggctgctcatgtgtcggtcctgtgggcggcgtctgccccggcaattctgccggagctgtggtttggtgttatgtgagccctgccgggaggcagaccatcagcctcctggccactgtacactccctgtcaaagaagcagctgaggagcggcgtcgggactttggagagaagttaactcgtctgcgggaacttatgggggagctgcagcggcggaaggcagccttggaaggtgtctccaaggaccttcaggcaaggtataaagcagttctccaggagtatgggcatgaggagcgcagggtccaggatgagctggctcgctctcggaagttcttcacaggctctttggctgaagttgagaagtccaatagtcaagtggtagaggagcagagttacctgcttaacattgcagaggtgcaggctgtgtctcgctgtgactacttcctggccaagatcaagcaggcagatgtagcactactggaggagacagctgatgaggaggagccagagctcactgccagcttgcctcgggagctcaccctgcaagatgtggagctccttaaggtaggtcatgttggccccctccaaattggacaagctgttaagaagccccggacagttaacgtggaagattcctgggccatggaggccacagcgtctgctgcctctacctctgttacttttagagagatggacatgagcccggaggaagtggttgccagccctagggcctcacctgctaaacagcggggtcctgaggcagcctccaatatccagcagtgcctctttctcaagaagatg

SEQ ID NO: 5 - DNA, 22 bp, unidentified
  source 1..22: mol_type = genomic DNA
cggaattcatgttcaatcttcc

SEQ ID NO: 6 - DNA, 24 bp, unidentified
  source 1..24: mol_type = genomic DNA
cctcgagtcatggggtggaatatc

SEQ ID NO: 7 - DNA, 24 bp, unidentified
  source 1..24: mol_type = genomic DNA
gactcagatctcgaggccaccatg

SEQ ID NO: 8 - DNA, 21 bp, unidentified
  source 1..21: mol_type = genomic DNA
cgggatcccattcctggagtg